(19) United States
(12) Patent Application Publication
(10) Pub. No.: US 2019/0142841 A1
(43) Pub. Date: May 16, 2019

(54) PHARMACEUTICAL COMPOSITIONS AND METHODS FOR ANESTHESIOLOGICAL APPLICATIONS

(71) Applicant: Harrow Health, Inc., San Diego, CA (US)

(72) Inventor: Dennis Elias Saadeh, Irvine, CA (US)

(21) Appl. No.: 16/250,450

(22) Filed: Jan. 17, 2019

Related U.S. Application Data
(63) Continuation-in-part of application No. 15/995,875, filed on Jun. 1, 2018.
Application No. 15/995,875 is a continuation-in-part of application No. 15/903,529, filed on Feb. 23, 2018, now Pat. No. 10,166,240.
Application No. 15/903,529 is a continuation-in-part of application No. 15/184,768, filed on Jun. 16, 2016, now Pat. No. 9,918,993.
Application No. 15/995,875 is a continuation-in-part of application No. 15/903,615, filed on Feb. 23, 2018, now Pat. No. 10,179,136.
Application No. 15/903,615 is a continuation-in-part of application No. 15/184,768, filed on Jun. 16, 2016, now Pat. No. 9,918,993.
(60) Provisional application No. 62/182,130, filed on Jun. 19, 2015.
(60) Provisional application No. 62/182,130, filed on Jun. 19, 2015.

(51) Int. Cl. A61K 31/5517; A61K 45/06; A61K 47/59; A61K 47/26; A61K 47/38; A61K 9/00; A61K 31/135; A61K 31/4178

(52) U.S. Cl. CPC A61K 31/5517; A61K 45/06; A61K 47/593; A61K 47/26; A61P 25/20; A61K 9/0053; A61K 31/135; A61K 31/4178; A61K 47/38

(57) ABSTRACT

Pharmaceutical compositions and methods for inducing conscious sedation using such compositions are described, the compositions including a benzodiazepine-based compound, an NMDA antagonist, and optionally a β-blocker, antiemetic, an NSAID, and/or an antihistamine medication. Compositions may be incorporated into vehicles for extended release. Methods for fabricating the compositions and using them for anesthesiological applications are also described.

### CROSS REFERENCE TO RELATED APPLICATION(S)

This is a continuation-in-part patent application claiming the benefit of priority under 35 U.S.C. § 120 to U.S. patent application Ser. No. 15/995,875, filed Jun. 1, 2018, now pending, which is a continuation-in-part of U.S. patent application Ser. No. 15/903,529, filed Feb. 23, 2018, now issued as U.S. Pat. No. 10,166,240, which is a continuation-in-part of U.S. patent application Ser. No. 15/184,768, filed Jun. 16, 2016, now issued as U.S. Pat. No. 9,918,993, which in turn claims the benefit of priority under 35 U.S.C. § 119(e) of U.S. provisional patent application No. 62/182,130, filed Jun. 19, 2015, the entire content of each of which is incorporated herein by reference. U.S. patent application Ser. No. 15/995,875, filed Jun. 1, 2018, is also a continuation-in-part claiming the benefit of priority under 35 U.S.C. § 120 to U.S. application Ser. No. 15/903,615, filed Feb. 23, 2018, now issued as U.S. Pat. No. 10,179,136, which is a continuation-in-part of Ser. No. 15/184,768, filed Jun. 16, 2016, now issued as U.S. Pat. No. 9,918,993, which claims the benefit of priority under 35 U.S.C. § 119(e) of U.S. Provisional App. No. 62/182,130, filed Jun. 19, 2015, the entire content of each of which is incorporated herein by reference.

### FIELD OF THE INVENTION

The present invention relates generally to the field of pharmacology and more specifically to compositions having anesthetic properties that are useful in various kinds of medical practice, such as surgery, and to methods of preparing and using such compositions.

### BACKGROUND

The present disclosure relates to solid or liquid pharmaceutical formulations comprising combinations of active agents such as anesthetics, anti-emetics, blood pressure, anti-anxiety medications and/or analgesics, and methods for using the same for providing anesthesia by administering such compositions orally, including such administrations as sublingual or buccal. The formulations may also include slow release reversal agents that would counteract the initial anesthesia effect.

It is necessary in many cases to use local anesthesia, particularly via oral route in the course of various surgical procedures, e.g., ophthalmic surgeries or urological interventions. For instance, when local anesthesia is employed during or prior to intraocular operations, the occurrences of pain, anxiety, peri-operative stress, nausea, agitation, vomiting and the like are less frequent, which will typically have a very beneficial effect on the surgical experience and reducing the number of intraocular complications such as bleeding, secretions, cardiac and/or pulmonary complications, etc. The severity of those complications when they do occur will also be less pronounced when local anesthesia is used.

Traditionally, an intravenous route is used to administer medications. Alternatives to intravenous methods and therapies have been suggested and previously used for the treatment. In particular, oral administration of benzodiazepines, opioid analgesics, propofol, ketamine or etomidate utilizing the MAC procedure (monitored anesthesia care) has been suggested and tried, but no more than minimal to moderate improvement has been achieved by such methods. Therefore, there remains a need for better treatments of these disorders.

This patent specification discloses such pharmaceutical compositions suitable for anesthesiological applications that can achieve positive patient outcomes while free of drawbacks and deficiencies of existing methods and formulations. Methods of fabricating and administering the same are also discussed.

### SUMMARY

According to one embodiment of the invention, there are provided pharmaceutical compositions. The compositions include a therapeutically effective quantity of at least one first pharmaceutically active compound comprising benzodiazepine moiety or pharmaceutically acceptable salts, hydrates, solvates or N-oxides thereof, a therapeutically effective quantity of at least one second pharmaceutically active compound that is an NMDA antagonist or pharmaceutically acceptable salts, hydrates, solvates or N-oxides thereof, and at least one pharmaceutically acceptable excipient or carrier therefor.

According to another embodiment of the invention, the pharmaceutical compositions described above may further include a therapeutically effective quantity of at least one third pharmaceutically active compound that is a β-blocker, a nonsteroidal anti-inflammatory drug (NSAID), or an antiemetic medicament, or a combination thereof, or pharmaceutically acceptable salts, hydrates, solvates or N-oxides thereof.

According to further embodiments of the invention, in the pharmaceutical compositions described above, the first pharmaceutically active compound may be any of midazolam, diazepam, lorazepam, flunitrazepam, alprazolam, chlordiazepoxide, clonazepam or clorazepate, the second pharmaceutically active compound may be any of ketamine, dextrorphan, etomidate, methadone, memantine, amantadine or dextromethorphan and the third pharmaceutically active compound may be (if a (β-blocker) any of metoprolol, propranolol, acebutolol, nadolol, atenolol, betaxolol, esmolol, bisoprolol fumarate, carvedilol, nebivolol, penbutolol, timolol, or sotalol or (if an antiemetic) ondansetron, dolasetron, granisetron, palonosetron, promethazine, imenhydrinate, or meclizine.

According to yet another embodiment of the invention, there are provided further pharmaceutical compositions such as any described above, wherein the compositions are formulated as a liquid or a solid item, e.g., a troche, a lozenge, a capsule, a pill, a cap and a bolus suitable for sublingual or oral administration.

According to other embodiments, there are provided specific compounds for making the compositions described above, for example, midazolam, ketamine and ondansetron, as well as methods for using above-mentioned composition(s) for the purposes of local anesthesia in various applications, such as ophthalmic surgery.

According to further embodiments of the invention, the above-mentioned methods of using the composition(s) include orally administering to a patient in need thereof (i.e., those patients who require conscious sedation or pre-sedation) a pharmaceutical composition described herein as the first step of a medical or surgical procedure, the procedure being an ophthalmic surgery (e.g., a cataract, glaucoma, corneal, eyelid surgery, or retinal surgery), a dental procedure (e.g., a tooth extraction, an oral surgery, or a root canal surgery), an outpatient medical procedure (e.g., medical imaging procedure, biopsy, bone marrow harvesting, colonoscopy, or endoscopy), a urological procedure, a laparoscopic procedure, obstetric and gynecological procedures, a gastrointestinal procedure, an otolaryngological procedure, a cosmetic surgery procedure, a dermatological procedure, a podiatric procedure, an orthopedic procedure, an emergency medical treatment, a psychiatric treatment, or a veterinarian procedure.

### DETAILED DESCRIPTION

### A. Terms and Definitions

Unless specific definitions are provided, the nomenclatures utilized in connection with, and the laboratory procedures and techniques of analytical chemistry, synthetic organic and inorganic chemistry described herein, are those known in the art. Standard chemical symbols are used interchangeably with the full names represented by such symbols. Thus, for example, the terms “hydrogen” and “H” are understood to have identical meaning. Standard techniques may be used for chemical syntheses, chemical analyses, formulating compositions and testing them. The foregoing techniques and procedures can be generally performed according to conventional methods well known in the art.

It is to be understood that both the foregoing general description and the following detailed description are exemplary and explanatory only and are not restrictive of the invention claimed. As used herein, the use of the singular includes the plural unless specifically stated otherwise. The section headings used herein are for organizational purposes only and are not to be construed as limiting the subject matter described.

As used herein, “or” means “and/or” unless stated otherwise. Furthermore, use of the term “including” as well as other forms, such as “includes,” and “included,” is not limiting.

“About” as used herein means that a number referred to as “about” comprises the recited number plus or minus 1-10% of that recited number. For example, “about” 100 degrees can mean 95-105 degrees or as few as 99-101 degrees depending on the context. Whenever it appears herein, a numerical range such as “1 to 20” refers to each integer in the given range; i.e., meaning only 1, only 2, only 3, etc., up to and including only 20.

The term “pharmaceutical composition” is defined as a chemical or biological compound or substance, or a mixture or combination of two or more such compounds or substances, intended for use in the medical diagnosis, cure, treatment, or prevention of disease or pathology.

The terms “anesthetic,” “anesthesia,” “anesthesiology” and the like refer herein to substances, compounds, processes or procedures that induce insensitivity to pain such as a temporary loss of sensation.

The term “conscious sedation,” which for the purposes of this application, may be used interchangeably with terms “procedural sedation” and “analgesia” is used herein to refer to an induced state of sedation characterized by a minimally depressed consciousness such that the patient is able to continuously and independently maintain a patent airway, retain protective reflexes, and remain responsive to verbal cues and/or tactile or physical stimulation.

Conscious sedation is typically performed/induced to decrease the level of anxiety in a patient and to elicit an improved degree of cooperation from the patient prior to or during a procedure. Conscious sedation, therefore, refers to a condition that is medically different and distinct from deep sedation which is the next level of sedation defined as depression of consciousness when the patient's ability to independently maintain ventilatory function may be impaired and he or she cannot be easily aroused; however, the patient will still purposefully respond to repeated or painful stimulation.

Conscious sedation is also clearly distinguishable for the purposes of the present application from the lower level of sedation (i.e., minimal sedation when the patient is able to maintain a normal response to verbal stimuli) as well as the highest level of sedation (i.e., general anesthesia when there is no response from the patient even with painful stimulus).

The term “pre-sedation” is defined for the purposes of this application as conscious sedation that is induced some time before a procedure, e.g., between about 5 minutes and about 1 hour prior.

The terms “solvate” and “hydrate” are used herein to indicate that a compound or substance is physically or chemically associated with a solvent for “solvates” such as water (for “hydrates”).

The term “NMDA antagonist” is defined as a compound that inhibits (“antagonizes”) the action of the N-methyl-D-aspartate receptors and is inclusive of both competitive and non-competitive antagonists, glycine antagonists and uncompetitive channel blockers, as these terms are understood by those having ordinary skill in the art.

The term “β-blocker” refers to a compound of any kind that can prevent or reduce the stimulation of the adrenergic receptors responsible for increased cardiac action.

The term “antiemetic” is defined as a drug or medicament that treats, reduces, and/or prevents nausea and/or vomiting.

The term “non-steroid anti-inflammatory drug” or “NSAID” refers to a class of compounds that are free of any steroid moieties yet are capable of providing analgesic, antipyretic and/or anti-inflammatory effects.

The term “antihistamine medicament” refers to any compound that is capable of inhibiting or counteracting the physiological effects of histamine.

The term “polyglycol” is defined as a polymer or oligomer containing several ether-glycol linkages that yields one or more glycols when these linkages are cleaved, e.g., by hydrolysis.

The term “carrier” refers to a substance that serves as a vehicle for improving the efficiency of delivery and the effectiveness of a pharmaceutical composition.

The term “excipient” refers to a pharmacologically inactive substance that is formulated in combination with the pharmacologically active ingredient of pharmaceutical composition and is inclusive of bulking agents, fillers, diluents and products used for facilitating drug absorption or solubility or for other pharmacokinetic considerations.

The term “binder” refers to a substance or compound that promotes, provides or improves cohesion, i.e., a substance that causes the components of a mixture to cohere to form a solid item that possesses integrity.

The term “troche” refers to a small tablet or lozenge (i.e., a medicated candy intended to be dissolved in the mouth), typically in a form of a disk, a ball or rhombic in cross-section, comprising medication and processed into a paste and dried.

The term “therapeutically effective amount” is defined as the amount of a compound or pharmaceutical composition that will elicit the biological or medical response of a tissue, system, animal or human that is being sought by the researcher, medical doctor or other clinician.

The term “pharmaceutically acceptable” when used in the context of a carrier, diluent or excipient, refers to a substance that is compatible with the other ingredients of the formulation and not deleterious to the recipient thereof.

The terms “administration of a composition” or “administering a composition” is defined to include an act of providing a compound of the invention or pharmaceutical composition to the subject in need of treatment.

The terms “oral administration” and “orally administering” are broadly defined as a route of administration where a medication is taken through the mouth including “sublingual administration” and “buccal administration” where the medication is placed under the tongue or between the gums and the cheek, respectively, to be absorbed by the body, or to be administered sublingually or buccally as a liquid.

### B. Embodiments of the Invention

According to embodiments of the present invention, there are provided pharmaceutical compositions for anesthetic purposes. The compositions comprise, consist of or consist essentially of, a combination of therapeutically effective quantities of at least one first pharmaceutically active compound and at least one second pharmaceutically active compound. In some further embodiments, the compositions optionally comprise, in addition to the above-mentioned first and second pharmaceutically active compounds, at least one third pharmaceutically active compound.

The first pharmaceutically active compound that is used in such composition comprises a benzodiazepine moiety or pharmaceutically acceptable salts, hydrates, solvates or N-oxides thereof. Those having ordinary skill in the art will know that benzodiazepine moiety is a structure where a benzene ring is condensed with diazepine ring, a seven-member heterocycle with two nitrogen atoms which for the purposes of this specification may be in any positions of the ring (e.g., 1,2-diazepine, 1,3-diazepine or 1,4-diazepine). An example of a compound having benzodiazepine moiety with 1,4-diazepine structure is shown below:

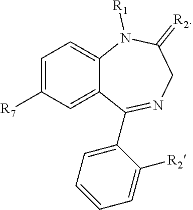

One particular first pharmaceutically active compound comprising a benzodiazepine moiety that can be used in pharmaceutical compositions described and claimed herein is midazolam. Other specific, non-limiting examples of first pharmaceutically active compounds comprising benzodiazepine moiety that can be used include diazepam, lorazepam, flunitrazepam, alprazolam, chlordiazepoxide, clonazepam, clobazam, bromazepam, prazepam, oxazepam and clorazepate. Each of these is also known under one or several trade names as shown in Table 1, which also discloses chemical names of such compounds. Those having ordinary skill in the art can select alternative suitable benzodiazepine-based compound for using in the compositions, if so desired.

TABLE 1
Examples of Benzodiazepine-Based Compounds That Can Be Used in Compositions

| Compound | Chemical Name (IUPAC) | Trade Name(s) |
|---|---|---|
| Midazolam | 8-chloro-6-(2-fluorophenyl)-1-methyl-4H- | VERSED ®, DORMICUM ®, |
| | imidazo[1,5-a][1,4]benzodiazepine | HYPNOVEL ® |
| Diazepam | 7-chloro-1-methyl-5-phenyl-3H-1,4- | VALIUM ®, DIASTAT ® |
| | benzodiazepin-2-one | |
| Lorazepam | 7-chloro-5-(2-chlorophenyl)-3-hydroxy- | TEMESTA ®, ATIVAN ®, |
| | 1,3-dihydro-2H-1,4-benzodiazepin-2-one | ORFIDAL ® |
| Flunitrazepam | 5-(2-fluorophenyl)-1-methyl-7-nitro-1H- | ROHYPNOL ®, NARCOZEP ® |
| | benzo[e][1,4]diazepin-2(3H)-one | and many others |
| Alprazolam | 8-chloro-1-methyl-6-phenyl-4H- | XANAX ® |
| | [1,2,4]triazolo[4,3-a][1,4]benzodiazepine | |
| Chlordiazepoxide | 7-chloro-2-methylamino-5-phenyl-3H-1,4- | LIBRIUM ® |
| | benzodiazepine-4-oxide | |
| Clonazepam | 5-(2-chlorophenyl)-7-nitro-2,3-dihydro- | KLONOPIN ®, RIVOTRIL ® |
| | 1,4-benzodiazepin-2-one | and many others |
| Clorazepate | 7-chloro-2,3-dihydro-2-oxo-5-phenyl- | TRANXENE ® |
| | 1H-1,4-benzodiazepine-3-carboxylic acid | |
| Bromazepam | 7-bromo-5-(pyridin-2-yl)-1H- | LEXOTAN ®, LEXOTANIL ® |
| | benzo[e][1,4]diazepin-2(3H)-one | and many others |
| Oxazepam | 7-chloro-3-hydroxy-5-phenyl-2,3-dihydro- | ALEPAM ®, SERAX ® and |
| | 1H-1,4-benzodiazepine-2-one | many others |
| Clobazam | 7-chloro-1-methyl-5-phenyl-1,5- | URBANOL ®, FRISIUM ®, |
| | benzodiazepine-2,4(3H)-dione | ONFI ® |
| Prazepam | 7-chloro-1-(cyclopropylmethyl)-5-phenyl- | LYSANXIA ®, CENTRAX ® |
| | 1,3-dihydro-2H-1,4-benzodiazepin-2-one | and many others |

The therapeutically effective quantity of the benzodiazepine-based compound(s) in the entire pharmaceutical composition can be between about 0.2 mass % and about 5.0 mass % of the composition. In some embodiments, the therapeutically effective amount of the benzodiazepine-based compound(s) can be between about 1.0 mass % and about 3.0 mass %, for example, about 2.5 mass % of the composition.

In some applications a patient may be extra sensitive to benzodiazepines (e.g., may become excessively drowsy). For such patients, there are provided additional embodiments of the composition in which benzodiazepine(s)-containing pharmaceutical compositions described above, would additionally include a quantity of a receptor antagonist to benzodiazepines. Such a receptor antagonist would begin counteracting the effect of benzodiazepine after the surgical procedure is complete, in essence providing a slow release feature. A non-limiting example of this antagonist is flumazenil also known under trade names such as ANEXATE®, ROMAZICON® and others. The use of antagonists is also envisioned as a routine practice (i.e., not just for sensitive patients), for example, in situations when a larger than typical or usual dosage of benzodiazepines is medically indicated, or recommended, or necessary. In some further applications, benzodiazepine-based compounds may be used in combination with non-benzodiazepine based sedatives such as eszopiclone, ramelteon, zolpidem, or zaleplon.

The second pharmaceutically active compound that is used in such compositions is an NMDA antagonist, as defined hereinabove, or pharmaceutically acceptable salts, hydrates, solvates or N-oxides thereof. One particular second pharmaceutically active compound that can be used in the pharmaceutical compositions described and claimed herein is ketamine. Other specific, non-limiting examples of NMDA antagonists that can be used include dextrorphan, etomidate, methadone, memantine, amantadine and dextromethorphan. Each of these is also known under one or several trade names as shown in Table 2, which also discloses chemical names of such compounds. Those having ordinary skill in the art can select alternative suitable NMDA antagonists for using in the compositions, if so desired.

TABLE 2
Examples of NMDA Antagonists That Can Be Used in Compositions

| Compound | Chemical Name (IUPAC) | Trade Name(s) |
|---|---|---|
| Ketamine | 2-(2-chlorophenyl)-2-(methylamino)cyclohexanone | KETANEST ®, |
| | | KETASET ®, |
| | | KETALAR ® (HCl salt) |
| Dextrorphan | 17-methyl-9a,13a,14a-morphinan-3-ol | None |
| Etomidate | Ethyl-3-[(1R)-1-phenyl-ethyl]imidazole-5-carboxylate | AMIDATE ®, |
| | | HYPNOMIDATE ® |
| Methadone | 6-(dimethylamino)-4,4-diphenylheptan-3-one | DOLOPHINE ®, |
| | | AMIDONE ® and others |
| Memantine | 3,5-dimethyladamantan-1-amine | AKATINOL ®, |
| | | NAMENDA ® and others |
| Amantadine | Adamantan-1-amine | SYMMETREL ® |
| Dextromethorphan | (4bS,8aR,9S)-3-methoxy-11-methyl-6,7,8,8a,9,10- | ROBITUSSIN ®, |
| | hexahydro-5H-9,4b-(epiminoethano)phenanthrene | DELSYM ® and others |

The therapeutically effective quantity of the NMDA antagonist(s) in the entire pharmaceutical composition can be between about 1.0 mass % and about 10.0 mass % of the composition. In some embodiments, the therapeutically effective amount of the NMDA antagonist(s) can be between about 4.0 mass % and about 6.0 mass %, for example, about 5.0 mass % of the composition. Accordingly, in various embodiments, the combined quantities of both the benzodiazepine-based compound(s) and the NMDA antagonist(s), taken together, in the entire pharmaceutical composition can be between about 1.2 mass % and about 15.0 mass % of the composition, such as between about 3.0 mass % and about 12.0 mass %, for example, about 10.0 mass % of the composition.

As mentioned above, the compositions may further optionally include at least one third pharmaceutically active compound. In such embodiments, the third pharmaceutically active compound is a β-blocker, as defined hereinabove, or pharmaceutically acceptable salts, hydrates, solvates or N-oxides thereof, or alternatively, and α-2-adrenergic agonist or, as another alternative, a pain reliever. In addition to, or instead of, β-blockers, the third pharmaceutically active compound may also include an antiemetic medicament, as defined hereinabove, or pharmaceutically acceptable salts, hydrates, solvates or N-oxides thereof.

In yet another aspect, the third pharmaceutically active compound may include one or several non-steroid anti-inflammatory drug(s) (NSAIDs), as defined hereinabove. NSAID(s) may be so used in addition to, or instead of, β-blocker(s), and/or antiemetic(s). In a further aspect, the third pharmaceutically active compound may also include an antihistamine medicament, as defined hereinabove. Non-limiting examples of specific antihistamine medicaments that can be so used include, but are not limited to, any of hydroxyzine pamoate, hydroxyzine hydrochloride, diphenhydramine hydrochloride, meclizine, chlorpheniramine, clemastine, promethazine, or prochlorperazine, or any combination thereof. Again, antihistamine medicaments may be used in addition to, or instead of, any of the above-mentioned compounds that may be used as the third pharmaceutically active compound.

The therapeutically effective quantity of the third pharmaceutically active compound(s) in the entire pharmaceutical composition can be between about 0.1 mass % and about 5.0 mass % of the composition. In some embodiments, the therapeutic effective amount of the third pharmaceutically active compound(s) can be between about 1.0 mass % and about 4.0 mass %, for example, about 2.5 mass % of the composition.

One particular β-blocker that can be used as the third pharmaceutically active compound in pharmaceutical compositions described and claimed herein is metoprolol. Other specific, non-limiting examples of β-blockers or α-2-adrenergic agonists or pain relievers that can be used include, propranolol, acebutolol, nadolol, atenolol, betaxolol, esmolol, bisoprolol fumarate, carvedilol, nebivolol, penbutolol, timolol, sotalol, dexmedetomidine hydrochloride, and acetaminophen. Each of these is also known under one or several trade names as shown in Table 3, which also discloses chemical names of such compounds. Those having ordinary skill in the art can select alternative suitable β-blockers for using in the compositions, if so desired.

TABLE 3
Examples of β-Blockers That Can Be Used in Compositions

| Compound | Chemical Name (IUPAC) | Trade Name(s) |
|---|---|---|
| Metoprolol | 1-(isopropylamino)-3-[4-(2-methoxyethyl)phenoxy]propan-2-ol | LOPRESSOR ®, |
| | | TOPROL ® |
| Propranolol | 1-(1-methylethylamino)-3-(1-naphthyloxy)propan-2-ol | CIPLA ®, |
| | | INDERAL ® and |
| | | many others |
| Acebutolol | N-{3-acetyl-4-[2-hydroxy-3-(propan-2-ylamino) | SECTRAL ®, |
| | propoxy]phenyl}butanamide | PRENT ® |
| Nadolol | 5-{[3-(tert-butylamino)-2-hydroxypropyl]oxy}-1,2,3,4- | CORGARD ® |
| | tetrahydronaphthalene-2,3-diol | |
| Atenolol | 2-{4-[2-hydroxy-3-(propan-2-ylamino)propoxy] | TENORMIN ® |
| | phenyl}acetamide | |
| Betaxolol | 1-{4-[2-(cyclopropylmethoxy)ethyl]-phenoxy}-3- | KERLONE ®, |
| | (isopropylamino)propan-2-ol | BETOPTIC ® and |
| | | others |
| Esmolol | 3-{4-[2-hydroxy-3-(propan-2-ylamino)propoxy] | BREVIBLOC ® |
| | phenyl}propanoate | |
| Bisoprolol | 1-[4-[[2-(1-methylethoxy)ethoxy]methyl]phenoxy]-3[(1- | ZEBETA ® |
| fumarate | methylethyl)amino]-2-propanol-2-butenedioate | |
| Carvedilol | 3-(9H-carbazol-4-yloxy)-2-hydroxypropyl-2-(2- | COREG ®, |
| | methoxyphenoxy)ethylamine | CARVIL ® and |
| | | many other |
| Nebivolol | 2,2′-azanediylbis(1-(6-fluorochroman-2-yl)ethanol) | NEBILET ®, |
| | | BYSTOLIC ® |
| Penbutolol | 1-(tert-butylamino)-3-(2-cyclopentylphenoxy)propan-2-ol | LEVATOL ®, |
| | | LEVATOLOL ® and |
| | | many others |
| Timolol | 1-(tert-butylamino)-3-[(4-morpholin-4-yl-1,2,5-thiadiazol-3- | TIMOPTIC ®, |
| | yl)oxy]propan-2-ol | BETIMOL ® and |
| | | many others |
| Sotalol | N-{4-[1-hydroxy-2-(propan-2-ylamino)ethyl]phenyl} | BETAPACE ® and |
| | methanesulfonamide | others |

One particular antiemetic that can be used as the third pharmaceutically active compound in pharmaceutical compositions described and claimed herein is ondansetron. Other specific, non-limiting examples of antiemetics that can be used include dolasetron, granisetron, palonosetron, promethazine, imenhydrinate, and meclizine. Each of these is also known under one or several trade names as shown in Table 4, which also discloses chemical names of such compounds. Those having ordinary skill in the art can select alternative suitable antiemetics for using in the compositions, if so desired.

TABLE 4
Examples of Antiemetics That Can Be Used in Compositions

| Compound | Chemical Name (IUPAC) | Trade Name(s) |
|---|---|---|
| Ondansetron | (RS)-9-methyl-3-[(2-methyl-1H-imidazol-1-yl)methyl]-2,3- | ZOFRAN ®, |
| | dihydro-1H-carbazol-4(9H)-one | ONDISOLV ® |
| Dolasetron | (2α,6α,8α,9aβ)-octahydro-3-oxo-2,6-methano-2H-quinolizin- | ANZEMET ® |
| | 8-yl-1H-indole-3-carboxylate monomethanesulfonate, | |
| | monohydrate | |
| Granisetron | 1-methyl-N-((1R,3r,5S)-9-methyl-9-azabicyclo[3.3.1]nonan-3- | KYTRIL ® |
| | yl)-1H-indazole-3-carboxamide | |
| Palonosetron | (3aS)-2-[(3S)-1-azabicyclo[2.2.2]oct-3-yl]-2,3,3a,4,5,6- | ALOXI ® |
| | hexahydro-1H-benz[de]isoquinolin-1-one | |
| Promethazine | (RS)-N,N-dimethyl-1-(10H-phenothiazin-10-yl)propan-2- | PHENERGAN ® |
| | amine | |
| Dimenhydrinate | 2-benzhydryloxy-N,N-dimethylethanamine; 8-chloro-1,3- | DRAMAMINE ®, |
| | dimethyl-7H-purine-2,6-dione | GRAVOL ®, |
| | | VOMEX ®, many |
| | | others |
| Meclizine | (RS)-1-[(4-chlorophenyl)(phenyl)methyl]-4-(3- | BONINE ®, |
| | methylbenzyl)piperazine | BONAMINE ®, |
| | | ANTIVERT ®, many |
| | | others |

Therefore, in various embodiments, the combined quantities of all the pharmaceutically active compounds (i.e., the benzodiazepine-based compound(s), the NMDA antagonist(s), the β-blocker(s)), and/or the antiemetic(s) taken together, in the entire pharmaceutical composition can be between about 1.3 mass % and about 20.0 mass % of the composition, such as between about 3.0 mass % and about 12.0 mass %, for example, about 10.0 mass % of the composition. Those having ordinary skill in the art will determine the most appropriate quantities of each the pharmaceutically active compound that are within the above-mentioned ranges and that are most suitable for a particular patient. As a non-limiting guideline only, the following mass ratios between the pharmaceutically active compounds may be used (Table 5) for compositions where the benzodiazepine-based compound is midazolam, the NMDA antagonist is ketamine hydrochloride and the β-blocker is propanolol hydrochloride:

TABLE 5
Exemplary Mass Ratios between Midazolam, Ketamine Hydrochloride
and Propanolol Hydrochloride in the Compositions

| | | Ketamine | Propanolol |
|---|---|---|---|
| Ratios | Midazolam | Hydrochloride | Hydrochloride |
| Between about | 1 | 2 | 1 |
| and about | 1 | 10 | 1 |
| Such as between about | 1 | 4 | 1 |
| and about | 1 | 6 | 1 |
| For example | 1 | 5 | 1 |

In one specific embodiment, which is exemplary and non-limiting, for the composition having midozalam as the first pharmaceutically active compound, ketamine as the second pharmaceutically active compound and ondansetron at the third pharmaceutically active compound, the mass midazolam:ketamine:odansetron ratio may be about 3:25:2.

The pharmaceutical compositions described herein may contain not only pharmaceutically active components but also, in some embodiments, may further include one or several inactive, neutral compounds which can be pharmaceutically acceptable excipient(s) or carrier(s), including, but not limited to, binder(s), antioxidant(s), adjuvant(s), synergist(s) and/or preservative(s). The mass concentration of such inactive compounds can be between about 80 mass % and about 99 mass % of the entire pharmaceutical composition, such as between about 85 mass % and about 95 mass %, e.g., about 90 mass %.

Some embodiments of the invention are directed to pharmaceutical formulations that are formulated as solid articles suitable for sublingual or oral administration, such as troches, lozenges, capsules, pills, caps or boluses. These solid compositions typically comprise binder(s) and/or excipient(s). They can be prepared by first mixing the pharmaceutically active compounds described above with suitable binder(s) and/or excipient(s) followed by molding or compressing the blend. Both hard and chewable lozenges and troches are within the scope of the invention.

Typical binder(s) that can be used for fabricating solid articles mentioned above include, without limitation, polyglycols as defined above, such as, e.g., polyethylene glycols (PEGs), polyethylene oxides (POE), methoxypolyethylene glycols, polypropylene glycols, polybutylene glycols or derivatives thereof having a molecular weight that is sufficient to provide the necessary hardness and time for dissolution of the troche; for example, the acceptable molecular weight can be within the range of between about 1,000 Daltons and about 8,000 Daltons. In some embodiments PEG-1450 or PEG-400 can be used. Non-limiting examples of some specific polyglycol derivatives that can be used are:
(a) PEG-laureates and dilaureates (e.g., PEG-10-, PEG-12-, PEG-20-, PEG-32-laurates, PEG-20- and PEG-32-dilaurates, PEG-20-glyceryl-, PEG-30-glyceryl- and PEG-40-glyceryl-laurates, PEG-80-sorbitan laurate);
(b) PEG-oleates, dioleates and trioleates (e.g., PEG-12-, PEG-15-, PEG-20-, PEG-32, PEG-200- and PEG-400-oleates, PEG-20- and PEG-32- dioleates, PEG-20-trioleate, PEG-25-glyceryl trioleate, PEG-20-glyceryl- and PEG-30-glyceryl-oleates, PEG-40-sorbitan oleate);
(c) PEG-stearates and distearates (e.g., PEG-15-, PEG-40-, PEG-100-stearates, PEG-32-distearate and PEG-20-glyceryl stearate);
(d) castor, palm kernel, corn and soya oil derivatives of PEG (e.g., PEG-35-, PEG-40- and PEG-60-castor oils, PEG-40-, PEG-50- and PEG-60-hydrogenated castor oils, PEG-40-palm kernel oil, PEG-60-corn oil, PEG-30-soya sterol);
(e) other PEG derivatives (e.g., PEG-24- and PEG-30-cholesterol, PEG-25-phytosterol, PEG-6- and PEG-8-caprate/caprylate glycerides, tocopheryl PEG-100 succinate, PEG-15-100 octylphenol products and PEG-10-100 nonylphenol products);
(f) other products such as polyglyceryl-10-laurate, POE-9- and POE-23-lauryl ethers, POE-10- and POE-20-oleyl ethers, POE-20-stearyl ether, polysorbates-20 and 80, polyglyceryl-10-oleate, Tween 40, Tween 60, sucrose monostearate, monolaurate and monopalmitate and various products of Poloxamer series.

Typical excipient(s) that can be used for fabricating solid articles mentioned above include, without limitation, gelatin, sodium saccharin, stevioside, peppermint oil, or any natural or artificial fruit, vegetable, flower, beverage or candy flavor.

As stated above, the compositions may optionally further include one or several antioxidant(s). If antioxidant(s) are used, non-limiting examples of those that can be used include α-tocopherol acetate, acetone sodium bisulfite, acetylcysteine, ascorbic acid, ascorbyl palmitate, butylated hydroxyanisole, butylated hydroxytoluene, cysteine, cysteine hydrochloride, tocopherol natural, tocopherol synthetic, dithiothreitol, monothioglycerol, nordihydroguaiaretic acid, propyl gallate, sodium bisulfite, sodium formaldehyde sulfoxylate, sodium metabisulfite, sodium sulfite, sodium thiosulfate, thiourea and tocopherols.

As stated above, the compositions may optionally further include one or several adjuvant(s) or synergists(s). If adjuvant(s) or synergists(s) are used, non-limiting examples of those that can be used include citric acid, EDTA (ethylenediaminetetraacetate) and salts, hydroxyquinoline sulfate, phosphoric acid and tartaric acid.

As stated above, the compositions may optionally further include one or several preservative(s). If preservative(s) are used, non-limiting examples of those that can be used include benzalkonium chloride, benzethonium chloride, benzoic acid and salts, benzyl alcohol, boric acid and salts, cetylpyridinium chloride, cetyltrimethyl ammonium bromide, chlorobutanol, chlorocresol, chorhexidine gluconate or chlorhexidine acetate, cresol, ethanol, imidazolidinyl urea, metacresol, methylparaben, nitromersol, o-phenyl phenol, parabens, phenol, phenylmercuric acetate/nitrate, propylparaben, sodium benzoate, sorbic acids and salts, β-phenylethyl alcohol and thimerosal.

Accordingly, the pharmaceutical formulation can be administered to a subject in need of conscious sedation, procedural sedation, analgesia and/or pre-sedation, and in general for any kind of non-general anesthesia, by various local administrations. More specifically, the pharmaceutical formulations described herein may be prescribed by ordinarily skilled medical practitioners such as physicians, as the means of conscious sedation or pre-sedation. This is intended to be used for certain patients who experience or expect to experience high anxiety, bouts of panic attacks, disquietude, apprehension, angst or similar feelings of psychological discomfort or distress prior to, or during, medical or surgical procedures as described in more detail below. The patients may be of any age, i.e., including children, adolescents and adults.

For example, the formulation can be used prior to various outpatient surgeries and medical procedures, both invasive and non-invasive, such as an ophthalmic surgery, outpatient medical or surgical procedures, dental procedures, urological procedures, obstetric and gynecological procedures, gastrointestinal procedures, otolaryngological procedures, cosmetic surgery procedures, dermatological procedures, podiatric procedures, orthopedic procedures, emergency medical treatments, psychiatric treatments, and veterinarian procedures.

Specific representative examples of the procedures that are amenable to use of the formulation include, without limitation, cataract surgery, glaucoma surgery, corneal surgery, eyelid surgery, retinal surgery, tooth extraction, oral surgery, root canal surgery, medical imaging procedures (e.g., MRI or CAT scanning, especially for patients suffering from claustrophobia), biopsy, bone marrow harvesting, colonoscopy, endoscopy and laparoscopy.

In one non-limiting embodiment, the local administration is by oral route, such as sublingually or buccally, typically being delivered to the patient via a solid delivery vehicle such as a troche, a lozenge, a capsule, a pill, a cap, and a bolus, as mentioned above. In an additional embodiment, the pharmaceutical composition may be formulated as a liquid item adapted for sublingual or buccal administration (in which case it will include all the pharmaceutically active compounds described above, but no pharmaceutically suitable binder); such liquid formulations may be delivered by any method to be selected by one having ordinary skill in the art of delivery of medications, e.g., via a syringe or a pipette. Such local administration may be used instead or intravenous administration or to complement the latter, as appropriate.

It will be understood by those having ordinary skill in the art that the specific dose level and frequency of dosage for any particular patient may be varied and will depend upon many factors including the activity of the specific compound employed, the metabolic stability and length of action of that compound, the age, body weight, general health, gender, diet and the severity of the particular condition being treated.

In additional embodiments, the pharmaceutical compositions of the present invention may be incorporated into vehicles allowing extended release of the compositions over a period of time. To achieve this effect, the compositions may be combined with polymers forming such vehicles. The product will be extended release capsules ensconcing or enveloping the pharmaceutical formulation, or alternatively, a matrix polymer structure holding the pharmaceutical formulation that is embedded into the matrix.

The vehicle carrying the pharmaceutical formulation may be configured to allow the gradual release of the pharmaceutical formulation over not less than about 12 hours, such as between about 12 hours and about 20 hours, for example, about 16 hours. The rate of release may be uniform throughout the entire period of release; alternatively, those having ordinary skill in the art may formulate the release vehicle in such a way as to allow different rates of release at different times, for example, faster release at the beginning of the process of release and slower at later stages, or vice versa, or in any other way that may be necessary.

The vehicle may be manufactured from any pharmaceutically acceptable polymer that is capable of releasing at least 95 mass % of the pharmaceutical formulation that the vehicle incorporates within the above-mentioned time periods, i.e., within not less than 12 hours, or 12-20 hours. In some embodiments, the vehicle may be formulated to ensure the release of at least 97 mass % of the pharmaceutical formulation, for example, at least 99.5 mass %.

Those having ordinary skill in the art will select the most appropriate polymer for making the vehicle. As guidance only, some non-limiting examples of such polymers include, but are not limited to, esters of cellulose, e.g., methyl cellulose and hydroxypropyl methyl cellulose. Other acceptable polymers include, but are not limited to, poly(lactic-co-glycolic acid) (PLGA), polylactic acid, polyglycolide, dextrin, polyacetals, poly(N-(2-hydroxypropyl)methacrylamide), polycaprolactone, and poly-3-hydroxybutyrate.

One particular type of product that can be used in fabricating the vehicle carrying the pharmaceutical formulation may be water-soluble methylcellulose and hydroxypropyl methylcellulose polymers, such as METHOCEL® family of products, for example, a hydroxypropyl methylcellulose product METHOCEL® E4M, 20% METHOCEL® K4M, or 10% METHOCEL® K100 or, alternatively and particularly useful for hot melt extrusion, another hydroxypropyl methylcellulose product AFFINISOL™ HPMC (all mentioned hydroxypropyl methylcellulose polymers are available from Dow Chemical Co., Midland, Mich.).

While all the products that include vehicles carrying the pharmaceutical compositions of the present invention are useful for treating all the medical, surgical and other procedures mentioned above, those having ordinary skill in the art may find these systems particularly suitable and advantageous in the treatments of depression (including major depression or treatment-resistant depression), PTSD, alcohol or substance abuse/dependence, suicide prevention, anxiety (including generalized anxiety disorder), personality disorders (inclusive of borderline personality disorder), and related psychiatric and/or psychological disorders, syndromes, symptoms, maladies, and the like.

According to further embodiments, methods for fabricating the above-described pharmaceutical compositions are provided. A one-batch formulation method may be used, where the components of the pharmaceutical formulation can be combined in single container; the components may be added to the container simultaneously or consecutively. Alternatively, a two- or multiple-batch method(s) may be used if desired, where each component of the pharmaceutical formulation can be combined in separate container followed by combining the contents of each container.

In one exemplary, non-limiting procedure, pre-measured quantities of each ingredient in the form of dry powder can be mixed to form a dry blend followed by mixing it with a pre-molten troche base. The composition can then be molded to form a troche.

In additional embodiments, pharmaceutical kits are provided. The kit includes a sealed container approved for the storage of solid pharmaceutical compositions, the container containing one of the above-described pharmaceutical compositions an instruction for the use of the composition and the information about the composition are to be affixed to the container or otherwise enclosed with it.

The following examples are provided to further elucidate the advantages and features of the present invention, but are not intended to limit the scope of the invention. The examples are for the illustrative purposes only. USP pharmaceutical grade products were used in preparing the formulations described below.

### EXAMPLE 1

### Preparing a Pharmaceutical Composition in the Form of a Troche

A pharmaceutical composition may be prepared as described below. The following products can be used in the amounts and concentrations specified:
(a) about 0.2 g of midazolam;
(b) about 2.0 g of ketamine hydrochloride;
(c) about 0.2 g of ondansetron hydrochloride;
(d) about 1 mL of lemon oil flavoring; and
(e) about 15.5 g of standard troche base (comprising polyglycol 1450, polyglycol 400, gelatin, sodium saccharin and steviaside).

The troche base can be melted at low heat while being stirred; when completely molten, the heat can be turned off with continued stirring. All the dry ingredients, pre-weighed can be added into the molten base followed by adding the flavoring and mixing all components together.

While any shape may be used, a half-moon shaped troche mold can be lightly sprayed with PAM® (or a suitable oil/releasing agent) to cover the entire surface of the mold and the mixture prepared as explained above can then be poured into the mold and allowed to cool and harden at room temperature. A heat gun can then be used to smooth out the surface followed by another round of cooling at room temperature followed by removing the so prepared troche from the mold, placing it into a prescription vial and labeling the vial. The troche is now ready to be administered.

### EXAMPLE 2

### Preparing an Extended Release Pharmaceutical Composition

An extended release pharmaceutical composition may be prepared as described below. The following products can be used in the amounts and concentrations specified:
(a) about 3.0 g of midazolam;
(b) about 10.0 g of ketamine hydrochloride;
(c) about 21.0 g of Methocell® K100M USP grade powder;
(d) about 100 g of Methocell® E4M premium USP grade powder;
(e) about 15.0 g of microcrystalline cellulose powder; and
(f) 100 clear gelatin capsules size No. 0.

Midazolam and ketamine hydrochloride may be triturated to a fine powdery constituency using standard mortar and pestle. Using the principles of geometric dilution, the rest of ingredients (i.e., the two Methocell® powders and the microcrystalline cellulose powder) may be then mixed in with trituration in the mortar. The use of a V-blender and a powder food coloring may be employed to verify that the mixture is homogenous followed by sieving through an 80 mesh sieve to ensure the evenness or the particle sizes which can then be encapsulated in the clear gelatin capsules (size 0).

Principles of geometric dilution that should be followed, as mentioned above, are well known in the art, but for further guidance may be summarized as follows. A portion of an ingredient of a large quantity (L) is to be mixed into an ingredient of a smaller quantity (S) in small portions. To that end, first a portion of L is to be mixed with a portion of S, the two portions having the same volume as a portion of S thus obtaining a mixture M1. Then another portion of L is to be mixed with a portion of M1, the two portions having the same volume. This process is to be continued until the entire quantity of L is used up.

The entire quality of L is not to be added to the entire quantity of S in the expectation that the uniform dispersion can be more expeditiously obtained after brief trituration of the mixture.

Although the invention has been described with reference to the above examples, it will be understood that modifications and variations are encompassed within the spirit and scope of the invention. Accordingly, the invention is limited only by the following claims.

What is claimed is:

1. A pharmaceutical composition, comprising a therapeutically effective quantity of a pharmaceutical formulation incorporated into a vehicle, wherein the pharmaceutical composition comprises:
(a) a therapeutically effective quantity of a first pharmaceutically active compound selected from the group consisting of midazolam, diazepam, lorazepam, flunitrazepam, alprazolam, chlordiazepoxide, clonazepam and clorazepate, and pharmaceutically acceptable salts, hydrates, solvates or N-oxides thereof, optionally in combination with a non-benzodiazepine compound selected from the group consisting of eszopiclone, ramelteon, zolpidem, and zaleplon;
(b) a therapeutically effective quantity of a second pharmaceutically active compound selected from the group consisting of ketamine, dextrorphan, etomidate, methadone, memantine, amantadine, dextromethorphan, and pharmaceutically acceptable salts, hydrates, solvates or N-oxides thereof; and
(c) optionally, a pharmaceutically acceptable excipient,
wherein the vehicle is configured to provide extended release of the pharmaceutical formulation and is selected from the group consisting of extended release capsules ensconcing the pharmaceutical formulation and a matrix polymer structure holding the pharmaceutical formulation that is embedded into the matrix.

2. The pharmaceutical composition of claim 1, wherein the pharmaceutical formulation further comprises a therapeutically effective quantity of a third pharmaceutically active compound selected from the group consisting of β-blockers, antiemetic medicaments, NSAIDs, antihistamines, α-2-adrenergic agonists, pain relievers and combinations thereof, or pharmaceutically acceptable salts, hydrates, solvates or N-oxides thereof.

3. The pharmaceutical composition of claim 2, wherein the β-blocker, the α-2-adrenergic agonist or the pain reliever is selected from the group consisting of metoprolol, propranolol, acebutolol, nadolol, atenolol, betaxolol, esmolol, bisoprolol fumarate, carvedilol, nebivolol, penbutolol, timolol, sotalol, dexmedetomidine hydrochloride, and acetaminophen.

4. The pharmaceutical composition of claim 2, wherein the antiemetic medicament is selected from the group consisting of ondansetron, dolasetron, granisetron, palonosetron, promethazine, imenhydrinate, and meclizine.

5. The pharmaceutical composition of claim 2, wherein the NSAID is selected from the group consisting of bromfenac, ketorolac, etodolac, sulindac, diclofenac, aceclofenac, nepafenac, tolmetin, indomethacin, nabumetone, ketoprofen, dexketoprofen, ibuprofen, flurbiprofen, dexibuprofen, fenoprofen, loxoprofen, oxaprozin, naproxen, aspirin, salicylic acid, diflunisal, salsalate, mefenamic acid, meclofenamic acid, flufenamic acid, tolfenamic acid, meloxicam, piroxicam, ternoxicam, droxicam, lornoxicam, isoxicam, celecoxib, rofecoxib, valdecoxib, parecoxib, lumiracoxib, etoricoxib, firocoxib, nimesulide, clonixin, and licofelone.

6. The pharmaceutical composition of claim 2, wherein the antihistamine is selected from the group consisting of hydroxyzine pamoate, hydroxyzine hydrochloride, diphenhydramine hydrochloride, meclizine, chlorpheniramine, clemastine, promethazine, and prochlorperazine.

7. The pharmaceutical composition of claim 2, wherein the pharmaceutical formulation further comprises a therapeutically effective quantity of a receptor antagonist to benzodiazepines.

8. The pharmaceutical composition of claim 7, wherein the receptor antagonist is flumazenil.

9. The pharmaceutical composition of claim 2, wherein the first pharmaceutically active compound is midazolam, the second pharmaceutically active compound is ketamine and the third pharmaceutically active compound is metoprolol, wherein the midazolam:ketamine:metoprolol ratio is between about 1:2:1 and about 1:10:1 by mass.

10. The pharmaceutical composition of claim 2, wherein the first pharmaceutically active compound is midazolam, the second pharmaceutically active compound is ketamine and the third pharmaceutically active compound is ondansetron, wherein the midazolam:ketamine:ondansetron ratio is about 3:25:2 by mass.

11. The pharmaceutical composition of claim 1, wherein the vehicle is configured to allow the release of the pharmaceutical formulation in a period of time between about 12 hours and about 20 hours.

12. The pharmaceutical composition of claim 1, wherein the vehicle comprises a polymer selected from the group consisting of esters of cellulose, poly(lactic-co-glycolic acid), polylactic acid, polyglycolide, dextrin, polyacetals, poly(N-(2-hydroxypropyl)methacrylamide), polycaprolactone, and poly-3-hydroxybutyrate.

13. The pharmaceutical composition of claim 12, wherein the esters of cellulose are selected from the group consisting of methyl cellulose and hydroxypropyl methyl cellulose.

14. The pharmaceutical composition of claim 1, wherein the excipient is selected from the group consisting of gelatin, sodium saccharin, stevioside, peppermint oil, cherry flavor, lemon oil, raspberry flavor and combinations thereof.

15. A method for providing extended release of the pharmaceutical formulation of claim 1 to a patient in need thereof, the method comprising:
(a) preparing the pharmaceutical formulation;
(b) incorporating the pharmaceutical formulation into the vehicle; and
(c) orally administering the pharmaceutical formulation incorporated into the vehicle to a patient in need thereof,
thereby providing for extended release of the pharmaceutical formulation.
